(19) United States
(12) Patent Application Publication
(10) Pub. No.: US 2021/0330292 A1
(43) Pub. Date: Oct. 28, 2021

(54) SYSTEMS AND METHODS FOR FAST ACOUSTIC STEERING VIA TILTING ELECTROMECHANICAL REFLECTORS

(71) Applicants: The Board of Trustees of the University of Illinois, Urbana, IL (US); The Texas A&M University System, College Station, TX (US)

(72) Inventors: Pengfei Song, Champaign, IL (US); Zhijie Dong, Champaign, IL (US); Matthew R. Lowerison, Champaign, IL (US); Jun Zou, College Station, TX (US); Shuangliang Li, Bryan, TX (US)

(21) Appl. No.: 17/237,011

(22) Filed: Apr. 21, 2021

Related U.S. Application Data
(60) Provisional application No. 63/014,073, filed on Apr. 22, 2020.

(51) Int. Cl. A61B 8/00; A61B 8/14; G01S 15/89

(52) U.S. Cl. CPC A61B 8/4461; A61B 8/145; G01S 15/8931; G01S 15/8993; A61B 8/4281

(57) ABSTRACT

High volume-rate three-dimensional (“3D”) ultrasound imaging using fast acoustic steering via tilting electromechanical reflectors is described. Ultrasound beams are directed towards one or more tilting reflectors, which are scanned through a range of tilt angles in order to image a 3D field-of-view with a high volume rate.

### CROSS-REFERENCE TO RELATED APPLICATIONS

This application claims the benefit of U.S. Provisional Patent Application Ser. No. 63/014,073, filed on Apr. 22, 2020, and entitled “HIGH VOLUME-RATE THREE-DIMENSIONAL ULTRASOUND IMAGING.”

### BACKGROUND

Ultrasound has become the most commonly used clinical imaging modality owing to its safety, low cost, and portability. Its high imaging frame rate allows operators to perform clinical diagnosis in real time, enabling rapid screening and image-guided interventional procedures. However, conventional ultrasound can only provide a two-dimensional (“2D”) image for three-dimensional (“3D”) tissue structures. This leads to a high degree of operator dependence and uncertainty in image-guided procedures because radiological assessment, targeting and image quantifications are dependent on transducer placement and patient positioning. Furthermore, ultrasound operators must mentally integrate 3D anatomy during the scan, a skill that takes a substantial amount of training and is associated with poor inter-observer reproducibility.

Achieving reliable 3D ultrasound imaging would be advantageous and have significant clinical value. For instance, 3D ultrasound imaging may be used to provide a comprehensive evaluation of a targeted tissue and could effectively alleviate user/operator dependence of ultrasound. 3D ultrasound can also be useful to achieve advances in clinical applications including blood flow volume measurement, prenatal evaluation, imaging-guided interventions such as heart valve surgery, and for the realization of emerging US imaging techniques such as 3D shear wave elastography and 3D super-resolution ultrasound microvessel imaging.

For 3D ultrasound applications, it is highly beneficial to achieve a high imaging volume-rate (“VR”) with adequate imaging quality. For example, it is extremely challenging to image a beating heart or a blood vessel with fast-moving blood using a low VR. One technique to achieve high VR 3D ultrasound imaging is by using 2D ultrasound transducers that allow for 3D electronic scanning and beamforming. 2D ultrasound transducers, however, involve controls and communications with tens of thousands of transducer elements, which is technically difficult and expensive to fabricate and computationally challenging for real-time 3D imaging. As such, intricate strategies such as microbeamforming and parallel receive beamforming are needed to mitigate the issue of high element count of 2D arrays, which limits the VR and imaging quality.

On the other hand, 3D ultrasound imaging based on mechanically moving 1D ultrasound transducers (i.e., wobbler or sweeper transducers) offers a cheaper and more practical solution than using 2D transducers. However, because the wobbler transducers involve mechanically sweeping a 1D transducer across a wide range of tissue, the VR is very low. These approaches are also susceptible to tissue and operator motion and is, therefore, not suitable for imaging dynamic properties of the tissue such as cardiac motion and blood flow.

### SUMMARY OF THE DISCLOSURE

The present disclosure addresses the aforementioned drawbacks by providing an acoustic steering device that includes a housing and a first and second reflector arranged within the housing. The second reflector is arranged within the housing and relative to the first reflector such that ultrasound beams incident upon the first reflector are reflected onto the second reflector whereupon the ultrasound beams are reflected to exit the housing. At least one of the first reflector and the second reflector are tiltable and configured to tilt over a range of tilt angles responsive to a driving signal.

It is another aspect of the present disclosure to provide a three-dimensional ultrasound imaging system that includes an ultrasound transducer, a housing, a tilting reflector arranged within the housing, a redirecting reflector arranged within the housing, and a connector that is configured to couple the ultrasound transducer to the housing. The tilting reflector is configured to tilt through a range of tilt angles in order to steer ultrasound beams incident upon the tilting reflector towards the redirecting reflector where the ultrasound beams are reflected by the redirector reflector to exit the housing.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is an example acoustic steering device for use with an ultrasound system to provide fast acoustic steering via tilting electromechanical reflectors.

FIG. 2 illustrates an example housing that can form a part of the acoustic steering device shown in FIG. 1 according to some embodiments described in the present disclosure.

FIG. 3A shows an example tilting reflector assembly that can form a part of the acoustic steering device shown in FIG. 1 according to some embodiments described in the present disclosure.

FIG. 3B shows another example tilting reflector assembly, which includes a double-hinge (e.g., a slow hinge and a first hinge) design.

FIGS. 4A-4C show an example of a tilting reflector being steered through a single tilting cycle. FIG. 4A shows a fast-tilting mirror tilting angle plot in one tilting cycle at a 250 Hz tilting frequency. FIG. 4B is a schematic plot of an ultrasound transducer and fast-tilting mirror (tilting through a range of tilt angles corresponding to a single tilting cycle) as well as the reflected ultrasound beams being actively swept by the fast-tilting mirror. FIG. 4C illustrates a synchronization between ultrasound data acquisition (circles) and the fast-tilting mirror tilting position (solid curve).

FIG. 5A is an example of a fabricated redirecting mirror constructed from a single-crystal wafer supported by a supporting material (e.g., an acrylic plastic wedge).

FIG. 5B is an example housing that contains a redirecting mirror and a fast-tilting mirror.

FIGS. 6A and 6B are schematic examples of using two tilting reflectors in an acoustic steering device. FIG. 6A shows a setup where the tilting reflector on the left sweeps the incident ultrasound beam from the transducer, and the tilting reflector on the right remains still (i.e., redirecting only). FIG. 6B shows a setup where the two tilting reflectors both tilt with coordinated tilting angles (e.g., one tilting with clockwise direction while the other tilting with counterclockwise direction). The scanning range is augmented using this setup.

FIGS. 7A and 7B show an example connector that can be used to connect an ultrasound transducer to a housing of an acoustic steering device according to some embodiments described in the present disclosure.

FIG. 8 shows an example acoustic lens that can be used to focus ultrasound beam(s) in the elevational direction.

FIGS. 9A-9F show examples of using curved tilting and/or redirecting mirrors for acoustic focusing in the elevational dimension.

FIGS. 10A-10C show an example of using ultrasound data from multiple spatial locations to beamform a higher quality image (e.g., an image with improved elevational resolution).

FIG. 11 shows another example of using ultrasound data from multiple spatial locations to beamform a higher quality image.

FIGS. 12A-12F illustrate different sampling mechanisms for FASTER 3D imaging. FIGS. 12A-12C show an example where ultrasound sampling is evenly distributed temporally across one cycle of tilting reflector vibration (FIG. 12A), but due to the sinusoidal motion of the reflector the spatial location of the ultrasound sampling beam is not evenly distributed (FIG. 12B). As a result, the reconstructed image (FIG. 12C) has lower imaging pixel resolution (i.e., coarser appearance) in the middle portion. FIGS. 12D-12F show an example where ultrasound sampling is unevenly distributed temporally (FIG. 12D), so that the spatial distribution of ultrasound beam is even (FIG. 12E). As a result, the final reconstructed image (FIG. 12F) has a homogenous imaging pixel resolution in the elevational dimension.

FIG. 13A shows an example scheme for compounding plane wave imaging. Three different steering angles are transmitted consecutively.

FIG. 13B shows an example scheme for line-by-line scanning using either focused beams or weakly focused wide beams. Three different beams at three different lateral locations are transmitted in different reflector tilting cycles.

FIGS. 14A-14C illustrate the volume rate and number of sampled elevational positions of an example FASTER 3D imaging implementation.

FIGS. 15A-15E show an example of image reconstruction for FASTER 3D imaging. FIG. 15A shows the ultrasound sampling points (circles) and the underlying tilting reflector motion signal (solid curve). FIG. 15B shows the ultrasound beam position, where each line indicates a unique spatial sampling position sampled at the time indicated in FIG. 15A. FIG. 15C shows an example of the raw data (e.g., before reconstruction or scan conversion) of four point targets (cross-sections of thin wires).

FIG. 15D shows the same four wire targets after performing scan conversion. FIG. 15E shows the 3D rendering of the wire targets.

FIG. 16 is a schematic plot of spatiotemporal interpolation that upsamples and aligns the original data onto a new spatiotemporal grid.

FIG. 17 illustrates an example housing that can form a part of the acoustic steering device shown in FIG. 1, in which wires are integrated into the housing to provide calibration of the acoustic steering device, according to some embodiments described in the present disclosure.

FIG. 18 illustrates an example calibration method based on lasers and position sensitive diodes.

FIGS. 19A and 19B illustrate examples of performing photoacoustic imaging with the acoustic steering devices described in the present disclosure.

FIG. 20 illustrates an example of performing shear wave elastography using the FASTER systems and techniques described in the present disclosure.

FIG. 21 is a block diagram of an example ultrasound imaging system that can be used with the FASTER imaging device described in the present disclosure.

### DETAILED DESCRIPTION

Described here are systems and methods for high volume-rate three-dimensional (“3D”) ultrasound imaging using fast acoustic steering via tilting electromechanical reflectors, which may be referred to as “FASTER”. Advantageously, the systems and methods described in the present disclosure address challenges with conventional 3D ultrasound imaging, including high cost, suboptimal imaging quality, and low volume scan rate. In particular, the FASTER systems and methods are capable of high volume-rate (e.g., upwards of 500-1000 Hz, as compared to 0.2-20 Hz for conventional techniques) large field-of-view (“FOV”) 3D imaging with conventional one-dimensional (“1D”) transducers.

As one non-limiting example, the FASTER systems and methods can be implemented for obstetric and prenatal imaging applications. Additionally or alternatively, the FASTER systems and methods can be used for blood flow volume measurements, image-guided interventions (e.g., heart valve surgeries), 3D shear wave elastography, 3D super-resolution ultrasound microvessel imaging, and so on.

Embodiments of the present disclosure include 3D ultrasound imaging systems and methods. In certain embodiments, 3D ultrasound imaging systems and methods utilize ultrasound beams (e.g., unfocused plane waves or weakly focused wide beams) and a tilting reflector (e.g., a water-immersible micro-fabricated mirror). In certain embodiments, the 3D ultrasound imaging systems and methods may utilize ultrafast unfocused plane waves (e.g., 10-30 kHz) and a fast-tilting reflector (e.g., 250-500 Hz). Certain embodiments achieve a high imaging VR, such as an imaging VR in the range of 500-1000 Hz with a 3D FOV.

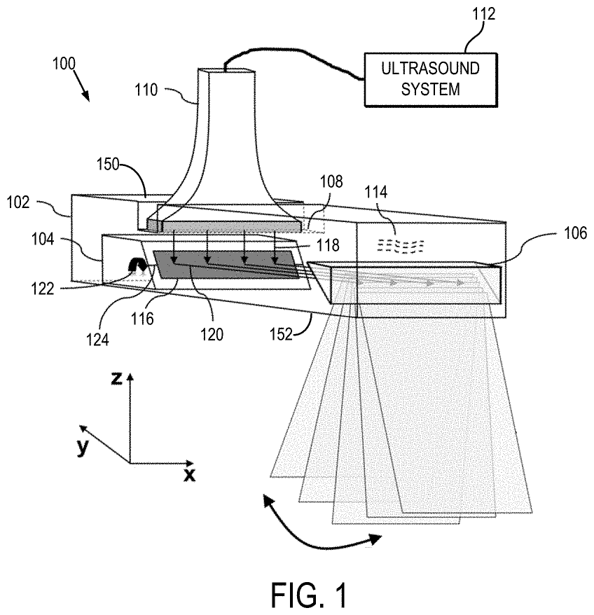

FIG. 1 illustrates an example three-dimensional ultrasound imaging system according to some embodiments of the present disclosure. The imaging system illustrated in FIG. 1 implements fast acoustic steering via tilting electromechanical reflectors (“FASTER”) device to facilitate high volume-rate 3D imaging.

Referring to FIG. 1, an acoustic steering device is shown as a FASTER imaging device 100 according to an embodiment of the present disclosure. The FASTER imaging device 100 generally includes a housing 102, a tilting reflector assembly 104, a redirecting reflector 106, and a connector 108 for connecting the housing 102 to an ultrasound transducer 110 of an ultrasound system 112. In FIG. 1, the coordinate axis indicates that coordinates of imaging dimensions used in image reconstruction. The x-axis indicates the lateral dimension, the z-axis indicates the axial dimension, and the y-axis indicates the elevational dimension. The imaging plane defined by “x-z” may be referred to as the azimuthal imaging plane, the imaging plane defined by “y-z” may be referred to as the elevational imaging plane, and the imaging plane defined by “x-y” may be referred to as the c-plane per ultrasound imaging convention.

In general, the ultrasound transducer is coupled to an upper surface 150 of the housing 102 and operated to generate ultrasound beams that are directed inwards towards the tilting reflector assembly 104, which constitutes a first reflector. The ultrasound beams incident upon the tilting reflector assembly 104 are then redirected to propagate along a direction within the housing 102 towards the redirecting reflector 106, which constitutes a second reflector. The ultrasound beams incident upon the redirecting reflector 106 are then redirected to propagate along a direction outward from the lower surface 152 of the housing 102 and into the tissue or other media of interest.

The housing 102 is an extension device that can be coupled or otherwise attached to any ultrasound transducer 110. In some configurations, the housing 102 may be filled with an acoustic conduction medium 114, such as water, gel (e.g., ultrasound gel), or oil. In these instances, the housing 102 may be sealed so that the acoustic conduction medium 114 does not leak out of the inner volume of the housing 102.

In general, the housing 102 is composed of biocompatible materials, thereby allowing it to be safely used in contact with humans and animals.

Advantageously, the housing 102 may also be sterilized for repeated use. As a non-limiting example, the housing 102 may be composed of one or more acoustically transparent materials, such as a thermoplastic elastomer (“TPE”), including a polyether block amide (e.g., PEBAX® manufactured by Arkema S.A. (Colombes, France)). In these configurations, the housing 102 does not need acoustic windows in order to conduct acoustic energy from the ultrasound transducer 110 to the components within the housing 102 and then to the targeted tissue medium. In other configurations, one or more acoustic windows may be implemented to facilitate the transmission of ultrasound energy through the housing 102. The shape of the housing 102 may be arbitrary or may be designed based on the principles of human factors and ergonomics.

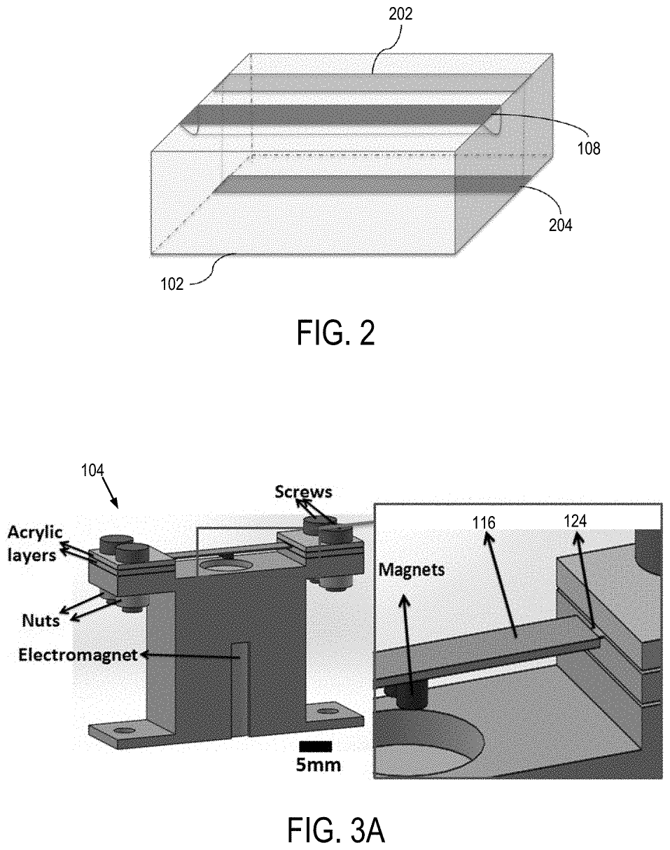

As shown in FIG. 2, in some embodiments the housing 102 can be designed in such a way to aid in scanning with the FASTER imaging device 100. In the example shown in FIG. 2, the housing 102 includes a visual guide 202 to help the operator align the underlying tissue anatomy with the actual ultrasound beam position. Because of the ultrasound beam reflections occurring inside the FASTER imaging device 100 (i.e., within the housing 102), the ultrasound beam position is no longer directly under the ultrasound transducer 110, which can be problematic for targeting the tissue during imaging. The visual guide 202 marks the position where the ultrasound beam enters the tissue and can be clearly visualized by the operator when using the FASTER imaging device 100.

The housing 102 may also include an acoustic window, or other acoustically transparent slot, 204 that allows the ultrasound beam to transmit through without significant attenuation or phase aberration before entering the tissue. The acoustic window 204 can be made by cutting an aperture at the bottom of the housing 102 that is large enough to let through the ultrasound beams swept at all angles and then sealed with materials such as TPX films (polymethylpentene) or plastic membranes that are acoustically transparent. The acoustic window 204 may also be integrated with the rest of the housing 102 (e.g., when the housing is constructed from an acoustically transparent material, such as PEBAX). When the housing 102 is constructed from an acoustically transparent material, then in some configurations no physical aperture needs to be created for the acoustic window 204.

In other instances, it may be advantageous to have the acoustic window 204, and/or the area on the housing 102 where the connector 108 is located, be thinner than the rest of the housing 102. Having these areas be thinner than the rest of the housing 102 can help alleviate acoustic attenuation, which may otherwise be present when the housing 102 is composed of a material such as PEBAX. In these instances, the acoustic window 204, and/or the area on the housing 102 where the connector 108 is located, is not an aperture, but a region on the housing 102 with thinner material. The thinner areas can be created, as an example, by removing a prescribed amount of material from the housing 102 in these locations.

The tilting reflector assembly 104 generally includes a tilting, or otherwise rotatable, reflector 116 that can steer, reflect, or otherwise redirect, ultrasound beams incident upon the reflector 116 from an incident direction 118 to a propagation direction 120. In some configurations, the tilting reflector assembly 104 may include a fast tilting reflector that can rapidly steer the incident ultrasound beams from the incident direction 118 to the propagation direction 120. As one non-limiting example, the orientation of the tilting reflector 116 can be changed at a rate of 250 Hz angular frequency.

In general, the tilting reflector assembly 104 may include at least one tilting reflector 116 that has an acoustic reflection coefficient sufficient to reflect or redirect the incident ultrasound beam(s) from the incident direction 118 to the propagation direction 120. The tilting reflector 116 can be fabricated as a microelectromechanical system (“MEMS”) mirror. As a non-limiting example, the tilting reflector 116 can be constructed from a single-crystal wafer (e.g., a polished single-crystal silicon), which has a reflection coefficient close to 1 (i.e., 100%) for acoustic waves. The tilting reflector 116 may tilt or otherwise rotate (as indicated by arrow 122) around a pivot 124, resulting in the incident ultrasound beam(s) being redirected from the incident direction 118 to the propagation direction 120. As an example, the pivot 124 may include one or more hinges.

As a non-limiting example, tilting reflector assembly 104 may include a tilting reflector 116 constructed as a single- or multi-facet reflective mirror mounted on a pivot 124 constructed as a rotational axle and driven by a micro-electrical motor. Alternatively, the tilting reflector assembly 104 may include a tilting reflector 116 fabricated by a micro-fabrication technique and realized by suspending a piece of a silicon mirror on top of a solenoid. Two small magnets with opposite polarities may then be positioned on the backside of the silicon mirror so that the mirror can be tilted responding to the input frequency and amplitude of an alternating current (“AC”) signal to the solenoid.

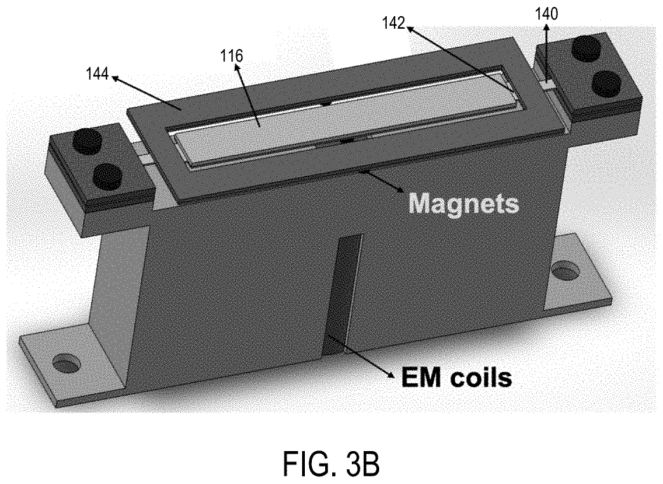

FIG. 3A illustrates an example tilting reflector assembly 104 manufactured in accordance with an embodiment of the present disclosure. Referring to FIG. 3A, the tilting reflector 116 may be a reflective mirror plate made of a polished wafer, which is made of a suitably hard material (e.g., single crystal silicon or quartz). The high acoustic impedance and the flatness of the polished wafer provide high acoustic reflectivity and little distortion to the reflected ultrasound beam. The length and width of the mirror plate may be made slightly larger than the size of the cross-section area of the ultrasound beam incident onto the mirror plate. The mirror plate may be supported by a pivot 124 that includes two rotational or torsional hinges made of flexible high-strength materials, such as polymers or metals. Therefore, the hinges can well withstand the possible impact damage due to the shock or turbulence in liquids. The two support hinges may be glued or mechanically clamped onto a holder by small screws.

To enable an underwater or a through-media scanning operation (e.g., when the housing 102 is filled with water or other acoustic coupling medium, such as gel or oil), electromagnetic actuation may be selected as the driving mechanism for tilting the tilting reflector 116 around the pivot 124 (i.e., torsional hinges). Compared with other actuation methods, electromagnetic actuation does not involve high voltages and therefore is more suitable for underwater operation. Magnet discs (e.g., two magnet discs with opposite polarity) may be attached to two symmetric positions around the rotating axis at the center of the mirror plate. An electromagnet coil may be assembled into the holder, which can be located directly underneath the magnetic discs. When a direct current (“DC”) or AC is passing through the electromagnet coil, the magnetic field generated by the electromagnet coil creates a torque on the magnet discs to tilt or vibrate the tilting reflector 116 around the pivot 124. As one non-limiting example, the tilting reflector 116 can be constructed as a micro-fabricated reflective mirror with an overall dimension of 40.2 mm (L) by 11 mm (W) by 30.2 mm (T).

As another example of the tilting reflector assembly 104, FIG. 3B shows a design of a double-axis fast-tilting mirror for 3D imaging with a wide range of volume rate and extended 3D FOV. In contrast to the design shown in FIG. 3A, where one pivot 124 (e.g., a single pair of stiff torsion hinges) is used to support the tilting reflector 116 (e.g., a reflection mirror plate), the double-axis design uses two sets of hinges—a first hinge pair 140 and a second hinge pair 142—to provide more imaging configuration flexibility and extending the imaging FOV. The single pivot 124 shown in FIG. 3A provides a limited resonance frequency peak around several hundred Hz. When the tilting reflector 116 is tilted back and forth around the resonance frequency, the maximal scanning angle and FOV will be achieved under the most energy-efficient driving conditions. If the tilting reflector (e.g., mirror) 116 is driven at a frequency far from its resonance, the achievable scanning angle, and therefore FOV, will be reduced.

In the double-axis design shown in FIG. 3B, two pairs of hinges are used. The first hinge pair 140 is coupled to a frame 144 (e.g., at the outer periphery of the frame 144) and the second hinge pair 142 couples the frame 144 (e.g., at the inner periphery of the frame 144) to the tilting reflector 116. The first hinge pair 140 may be referred to as a slow hinge and the second hinge pair 142 may be referred to as a fast hinge. The second hinge pair 142 (i.e., the fast hinge) and the frame 144 allow for the tilting frequency range to be extended. The first hinge pair 140 (i.e., the slow hinge) has a higher bending stiffness and lower torsional stiffness than the second hinge pair 142 (i.e., the fast hinge) and, therefore, provides a low and wide resonance frequency (e.g., 0-50 Hz, corresponding to a volume rate of 0-100 Hz). Because the resonance frequencies of the two pairs of hinges (i.e., slow and fast) are different, the tilting motion of the tilting reflector 116 and the frame 144 will be decoupled through the dynamic structural filtering effect, and therefore can be independently controlled with the two driving currents with different frequencies, as described by S. Xu, et al., in “A Two-Axis Water-Immersible Micro Scanning Mirror Driven by Single Inductor Coil through Dynamic Structural Filtering,” Sensors and Actuators A: Physical, 2018; 284: 172-180, which is herein incorporated by reference in its entirety.

With the double-axis design, the tilting reflector 116 can provide a myriad of reconfigurable scanning modes for enhancing the 3D imaging capability, as summarized below in Table 1.

TABLE 1
Summary of the Imaging Sequences and Imaging Modes of FASTER 3D-US

| | 3D B-mode | | 3D CFI/PD | | | | 3D SWE | | |
|---|---|---|---|---|---|---|---|---|---|
| | Scan | VR | Scan | Flow | VR | PRF | Scan | VR | PRF |
| Imaging method | mode | (Hz) | mode | measure | (Hz) | (Hz) | mode | (Hz) | (Hz) |
| Plane wave with | | | Fast | True-3D | 50-125 | 1000 | Fast | 1-2 | 2k-4k |
| coded excitation | | | Slow | Stacked 2D | 50-100 | 20k-30k | | | |
| Compounding plane wave | Fast/Slow | 50-200 | Slow | Stacked 2D | 10-20 | 4k-6k | Fast | 0.5-1 | 2k-4k |
| Line-by-line scanning | Fast/Slow | 10-100 | Slow | Stacked 2D | 2-4 | 20k-30k | | | |

(VR: volume rate; PRF: pulse repetition frequency; CFI: color flow imaging; PD: power Doppler; SWE: shear wave elastography; Slow: scanning with the pair of slow hinges; Fast: scanning with the pair of fast hinges)

For example, there are two basic scanning modes that use the two different types of supporting hinges. The first mode is the slow mode using the slow hinge. In this mode, the mirror tilting frequency is reduced to several Hz to several tens of Hz to allow adequate “dwelling” time at the same spatial locations to acquire data, which is useful for imaging applications such as color flow imaging and power Doppler where multiple Doppler ensembles need to be acquired with high PRF at each spatial location. The second scanning mode uses the fast hinge to quickly sweep the 3D volume at several hundred to several thousand Hz. This scanning mode can be used for shear wave elastography and blood flow imaging where a high-volume rate is advantageous.

The double-axis design shown in FIG. 3B also allows the tilting reflector 116 and frame 144 to be steered to an offset angle by applying a DC voltage. For example, the tilting reflector 116 and the frame 144 can be steered to an offset angle (e.g., from 0 degrees center location to an offset 15 degrees center location), and then the tilting reflector 116 can tilt around the new offset position (e.g., from −25 degrees-25 degrees around the 0 degree center location to −10 degrees to 40 degrees around the offset 15 degree center location). This feature effectively extends the FOV in the elevational imaging direction by giving the flexibility of positioning the FOV in arbitrary locations. 3D volumes acquired from different mirror offset angles can also be stitched together to construct a large FOV.

Referring again to FIG. 1, the tilting reflector assembly 104 may house a signal generator and a power source (e.g., a battery) to drive the tilting reflector 116. The signal generator may generate a driving signal that drives the tilting reflector 116. An example design of the signal generator may be based on a digital-to-analog converter with an amplifier that outputs a driving voltage at a specific frequency for driving the tilting reflector 116. In another embodiment, the driving signal may be supplied to the tilting reflector assembly 104 externally from the ultrasound system 112.

The driving signal (either from the tilting reflector assembly 104 or from the ultrasound system 112) may be synchronized with the ultrasound system 112 so that the imaging sequence can be synchronized with the motion of the tilting reflector. As a non-limiting example, the synchronization may be achieved by aligning the starting time of the first ultrasound transmission of a volume acquisition with the neutral position of the tilting reflector 116 (e.g., the zero-degree phase/angle position). In an alternative embodiment, synchronization can be achieved by retrospectively aligning ultrasound data acquisitions with the tilting reflector 116 position (i.e., phase/angle of the driving signal or readout of mirror position by a calibration position sensitive diode (“PSD”)). The synchronization signal may be communicated between the tilting reflector assembly 104 and the ultrasound system 112 either via a wired connection (e.g., a USB cable) or a wireless connection (e.g., via Bluetooth or Wi-Fi). The power source inside the tilting reflector assembly 104 may, for example, be a disposable or rechargeable battery. If rechargeable, charging can be done either wirelessly or via a wired connection, such as USB.

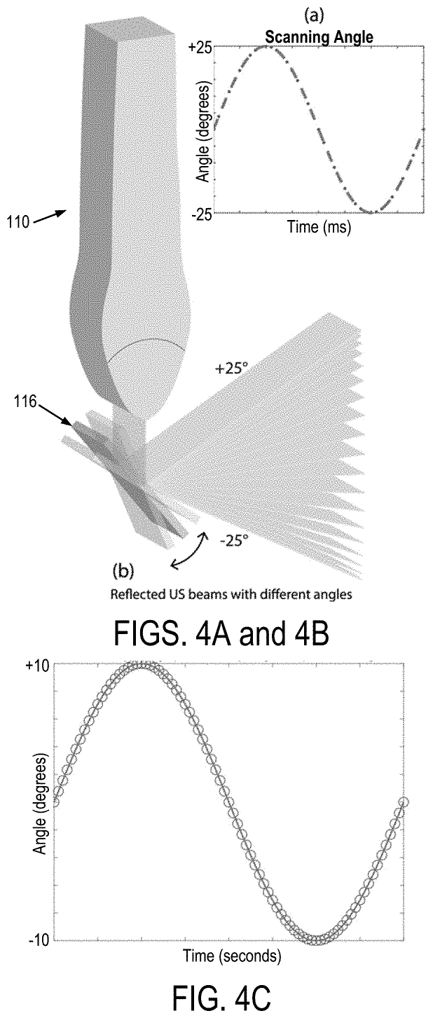

FIGS. 4A-4C illustrate an example tilting scan cycle using the FASTER imaging device 100. FIG. 4A shows an example plot of the scanning angle of the tilting reflector assembly 104 in one tilting cycle at a 250 Hz tilting frequency. FIG. 4B is a schematic plot of the ultrasound transducer and the tilting reflector 116 in the tilting reflector assembly 104 (tilting) as well as the reflected ultrasound (US) beams being actively swept by the tilting reflector assembly 104. FIG. 4C shows the synchronization between ultrasound data acquisition (blue circles) and the tilting position of the tilting reflector 116 in the tilting reflector assembly 104.

The redirecting reflector 106 may be a redirecting mirror that reflects the incident ultrasound beam(s) reflected off the tilting reflector 116 along the propagation direction 120 to propagate at a different direction. The redirecting reflector 106 allows for an upright position of the ultrasound transducer 110 so that the operator can use the ultrasound transducer 110 as they normally would. As a non-limiting example, redirecting reflector 106 may be made out of the same single crystal wafer as in the tilting reflector 116. The angle of the redirecting reflector 106 can be designed to direct the incident ultrasound beam(s) towards a desired direction. For example, if the incident ultrasound beam is horizontal, a 45-degree design for the redirecting reflector 106 can be used to redirect the incident beam to propagate in a vertical direction into the tissue. Note that, in some embodiments of the present disclosure, the redirecting reflector 106 (for redirecting the ultrasound beam) and the tilting reflector assembly 104 (for sweeping the ultrasound beam) can be interchanged; that is, the incident ultrasound beam from the ultrasound transducer 110 can be swept first by the tilting reflector assembly 104 and then redirected into the tissue by the redirecting reflector 106 (as shown in FIG. 1), or can be redirected first by the redirecting reflector 106 and then swept by the tilting reflector assembly 104 into the tissue.

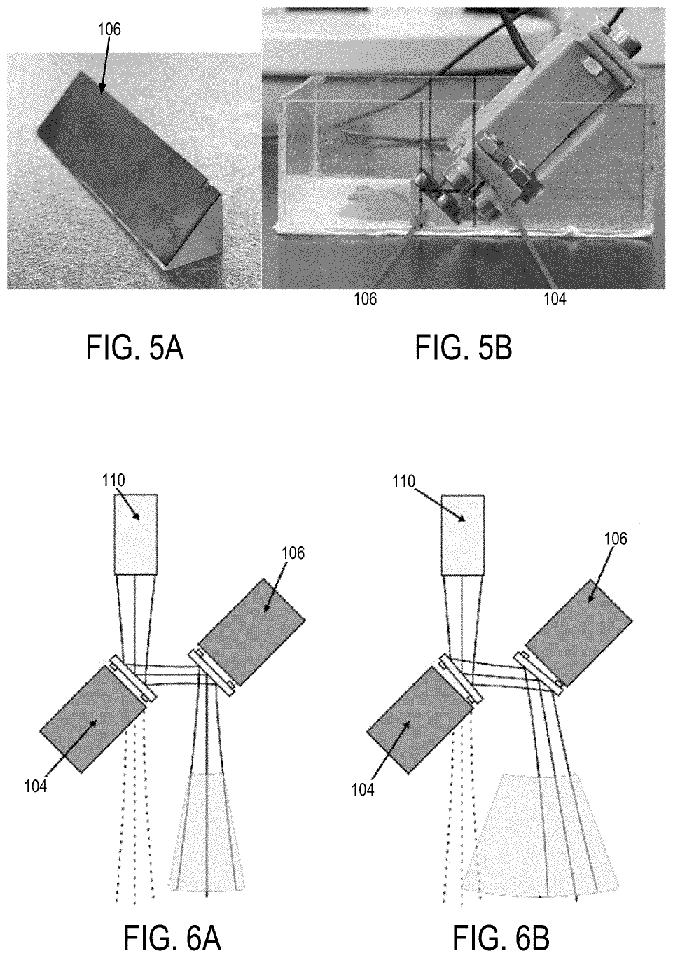

FIGS. 5A and 5B show an example of a fabricated redirecting reflector 106 that is made out of the same single-crystal wafer as in the fast-tilting mirror used in an example construction of the tilting reflector assembly 104. In this example, the redirecting reflector 106 is coupled to a supporting material composed of an acrylic plastic wedge. FIG. 5B shows an example device that holds the redirecting reflector 106 and the tilting reflector assembly 104 inside. In this example the ultrasound transducer would be positioned from the top and the incident beam will be redirected first by the redirecting mirror and then swept by the micro-fabricated mirror.

In some embodiments of the present disclosure, the redirecting reflector 106 may be another tilting reflector that is similar to the tilting reflector assembly 104. For instance, as illustrated in FIGS. 6A and 6B, with such a design, not only can the second tilting reflector redirect the ultrasound beam into the tissue to support an upright ultrasound position (FIG. 6A), but both tilting reflectors can be used to sweep the ultrasound beam. One advantage of using two tilting reflectors is the increased scanning range (i.e., larger field-of-view). For example, as shown in FIG. 6B, when the tilting angles of both reflectors are coordinated (e.g., one tilting reflector tilting clockwise while the other is tilting counterclockwise), then the effective scanning range can be expanded. When the two tilting reflectors both tilt but the tilting angles are opposed to each other (e.g., both tilting with clockwise or counterclockwise directions), then the effective scanning range will be reduced as compared to the scenarios shown in FIGS. 6A and 6B. Therefore, it is advantageous that the two tilting reflectors be temporally synchronized to tilt with coordinating angles in order to maximize the effective scanning range for 3D FASTER imaging.

The connector 108 provides a connection for the ultrasound transducer 110 to the housing 102 of the FASTER imaging device 100. The connector 108 allows the ultrasound transducer 110 to be firmly coupled or otherwise attached to the FASTER imaging device 100. Preferably, the attachment is strong enough to sustain the force and pressure generated from a combination of the ultrasound transducer 110 manipulation by the operator and subject's body during scanning. The connector 108 may also be configured to ensure that the ultrasound transducer 110 is aligned with the internal components of the FASTER imaging device 100 such as the tilting reflector assembly 104 and the redirecting reflector 106.

As non-limiting examples, the connector 108 can be built based on mechanical coupling (e.g., a “clip-on” mechanism, anchoring screws, adhesives, friction fit, full external housing), magnetic coupling (e.g., using magnets to attach the FASTER imaging device 100 to the ultrasound transducer 110), or other suitable connections that removably secure the ultrasound transducer 110 to the housing 102. In some embodiments, the connector 108 is integral with the housing 102. For instance, the connector 108 can be formed as a part of the housing 102. As an example, the connector 108 can be formed as an integral part of the housing 102 and provide for a mechanical or magnetic coupling of the ultrasound transducer 110 to the connector 108. In some other embodiments, the connector 108 can be a separate component that can be removably coupled to the housing 102. For instance, the connector 108 can itself be coupled to the housing 108 via mechanical coupling, magnetic coupling, or otherwise. As an example, the connector 108 can be mechanically coupled to the housing 102 via a clip-on mechanism, a snap-on mechanism, screws, or other mechanical connectors or fasteners.

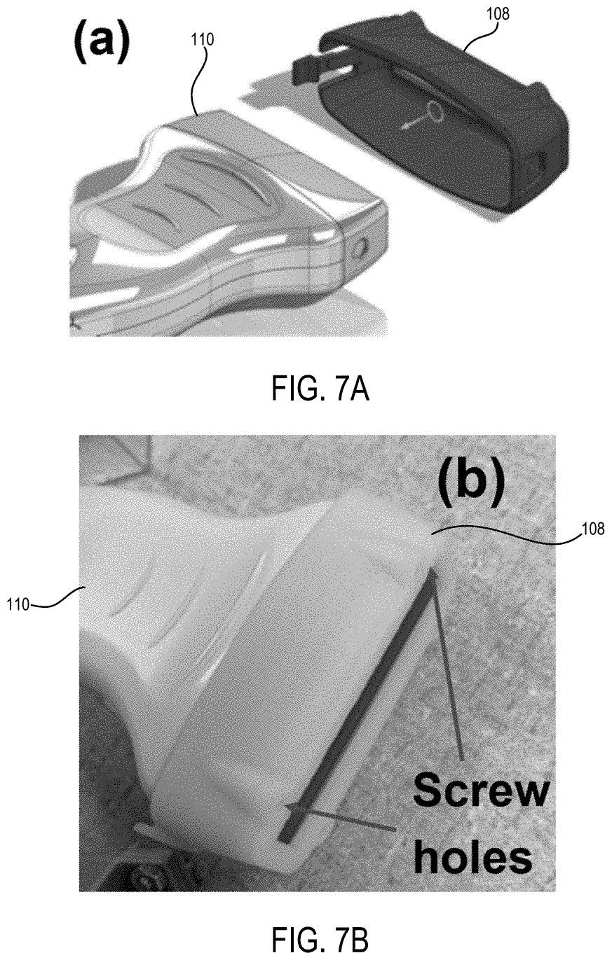

The connector 108 may be custom built to fit different ultrasound transducers from different manufacturers that have different exterior profiles. For example, FIGS. 7A and 7B show an example connector design that utilizes mechanical coupling to attach the FASTER imaging device 100 to the ultrasound transducer 110. The connector 108 can be custom designed and built to fit a specific ultrasound transducer 110 by using a clip-on mechanism. Additionally or alternatively, custom built screw holes can be made in the connector 108 to connect the ultrasound transducer 110 to the FASTER imaging device 100.

When an ultrasound transducer 110 is attached to the FASTER imaging device 100 via the connector 108, either an acoustic window (e.g., a membrane-sealed aperture) may be made to facilitate conduction of ultrasound waves into the FASTER imaging device 100 or the housing 102 may be composed of acoustically transparent materials, as described above, in order to make an intact surface with no acoustic windows. Ultrasound conduction gel can be applied in between the ultrasound transducer 110 and the surface of the FASTER imaging device 100.

The connector 108 can include a recessed region on the upper surface 150 of the housing 102, which is sized and shaped to receive the ultrasound transducer 110. Advantageously, the connector 108 can be configured to ensure acoustic beam alignment and/or sustain force and pressure during ultrasound scanning.

In some embodiments, the connector 108 may provide wired or wireless communication for components of the FASTER imaging device 100, the ultrasound system 112, or both. The connector 108 may also house a power source, such as a battery, to provide power for operation of the tilting reflector assembly 104. The connector 108 may in some instances be configured to charge such a battery. For example, the connector 108 may include one or more induction coils for wirelessly charging the battery.

The FASTER imaging device 100 can be configured for use with any suitable ultrasound transducer 110. For instance, in addition to using 1D ultrasound transducers, in embodiments of the present disclosure, the transducer 110 may be a 2D ultrasound transducer. Some non-limiting examples of 2D ultrasound transducers compatible with the FASTER 3D ultrasound imaging device 100 include 2D matrix arrays, row-column addressing arrays, and 2D transducers with arbitrary element positions (e.g., a sparse array). In the case of using a 2D ultrasound transducer with the FASTER 3D ultrasound imaging system 100, the device augments the 3D FOV of the 2D ultrasound transducer by sweeping the volumetric ultrasound beam and redirecting the beam to positions where electronic steering cannot reach. Different types of the ultrasound transducers (e.g., linear array, curved array, phase array) can also be used with the proposed device. Furthermore, so called 1.5D ultrasound transducers may be used with the FASTER 3D ultrasound imaging system 100, permitting high volume rate 3D imaging and/or elevational beam focusing.

More generally, the transducer 110 can include one or more of a 1D ultrasound transducers with different types such as linear array, curved array, and phase array; 2D ultrasound transducers such as 2D matrix array, row-column addressing array, and sparse array; and other types of ultrasound transducers such as 1.5D array, endocavity transducers, intracardiac transducers, and transesophageal transducers.

In some implementations, the ultrasound beam may diverge with depth because of a lack of focusing, for transmit, receive, or both, in the elevational direction. In these instances, spatial resolution and imaging penetration will be deteriorated. Because conventional 1D ultrasound transducers only have one physical element in the elevational dimension, no electronic focusing is possible.

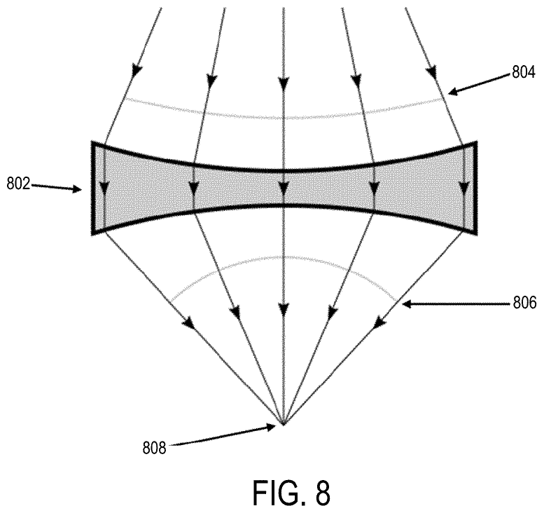

In some embodiments, an acoustic lens can be used to focus ultrasound in the elevational dimension, whether for transmit, receive, or both. As a non-limiting example, FIG. 8 shows a concave-shaped acoustic lens 802 that can refocus the diverged wave front 804 into a focused wave front 806 that is focused onto a focal point 808. Such an acoustic lens 802 can be used to focus the ultrasound beam in the elevational dimension. In one example, the acoustic lens 802 can be coupled to or otherwise arranged at the lower surface 152 of the housing 102 of the FASTER imaging device 100 (e.g., below where the ultrasound beam exits the housing 102 before entering the tissue), such that ultrasound beams redirected by the redirecting reflector 106 and exiting the housing 102 are focused before entering into the tissue or other media under examination. As another example, the acoustic lens 802 can be arranged within the housing 102 between the tilting reflector assembly 104 and the redirecting reflector 106. As still another example, the acoustic lens 802 can be arranged within the housing 102 between the connector 108 and the tilting reflector assembly 104, such that ultrasound beams entering the housing 102 are focused before impinging upon the tilting reflector assembly 104. In still other examples, one or more acoustic lenses 802 may be used in any combination of these locations or configurations.

As a non-limiting example, an acoustic lens 802 can be made out of a material such as a thermoplastic elastomer with a significantly higher ultrasound speed than soft tissue and water (e.g., a polyether block amide). Techniques such as 3D printing or mold casting can be used to fabricate the acoustic lens 802. In other non-limiting examples, one or more acoustic lens 802 could include a convex-shaped acoustic lens made out of a material with a significantly slower acoustic sound speed than soft tissue, a lens with a plano-convex lens shape, a lens with a plano-concave lens shape, a lens with a positive meniscus lens shape, a lens with a negative meniscus lens shape, and an adjustable acoustic lens designs (e.g., fluid inflatable membranes).

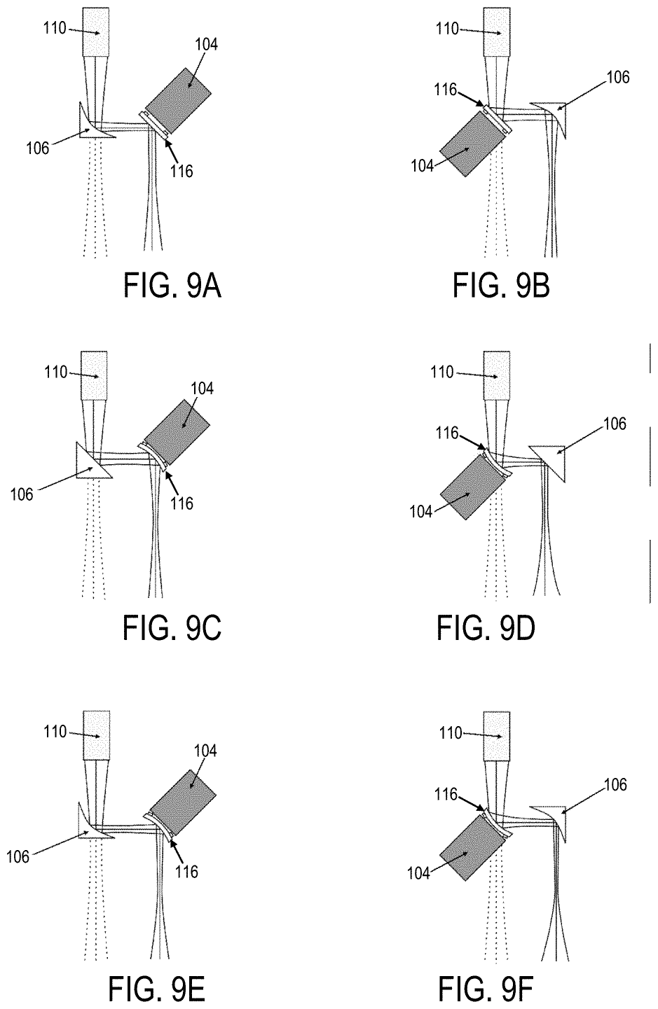

Additionally or alternatively, ultrasound beams can be focused by constructing the redirector reflector 106, the tilting reflector 116, or both as a curved reflector to focus the ultrasound beam in the elevational direction. Some non-limiting examples of concave reflector designs include spherical, parabolic, or hyperbolic shapes. FIG. 9A shows a design where the redirecting reflector 106 is concave and focuses the incident ultrasound beam from the ultrasound transducer 110 before being reflected by the tilting reflector assembly 104. FIG. 9B shows a similar design as FIG. 9A except that the positions of the tilting reflector assembly 104 and the redirecting reflector 106 are swapped. FIG. 9C shows a different design where a concave reflector is integrated into the tilting reflector 116 of the tilting reflector assembly 104 while the redirecting reflector 106 remains flat. In this configuration, the ultrasound beam gets steered and focused simultaneously by the tilting reflector assembly 104 before entering the tissue. FIG. 9D shows a similar design as FIG. 9C with the reflector positions swapped. FIGS. 9E and 9F show additional designs where both reflectors on the tilting and the redirecting component are concave, thereby both functioning as focusing reflectors. Again, the difference between FIGS. 9E and 9F is the reflector position. Note that the concave shaped reflectors can be used in combination with one or more acoustic lenses to maximize the focusing effect. The above examples are not limited to concave reflector designs only. For example, combinations of flat, concave, and convex reflectors can be used to correct for wave-front aberrations.

In addition to focusing the ultrasound beams as described above, the elevational resolution of FASTER 3D imaging can also be improved by using ultrasound beams with different directions, different scanning angles, or both to cover the same target in the FOV. In these instances, the ultrasound signal of the same target resulting from the multiple different ultrasound beams at multiple different spatial locations can be utilized to reconstruct the target image. For example, if the same target is detected by the ultrasound beam three times in one tilting cycle when the beam is steered at three locations, then the three sets of raw and unbeamformed ultrasound channel data can be used to beamform/reconstruct the 3D ultrasound data. This approach effectively increases the aperture size for beamforming, which narrows the mainlobe width and improves the imaging resolution in the elevational direction.

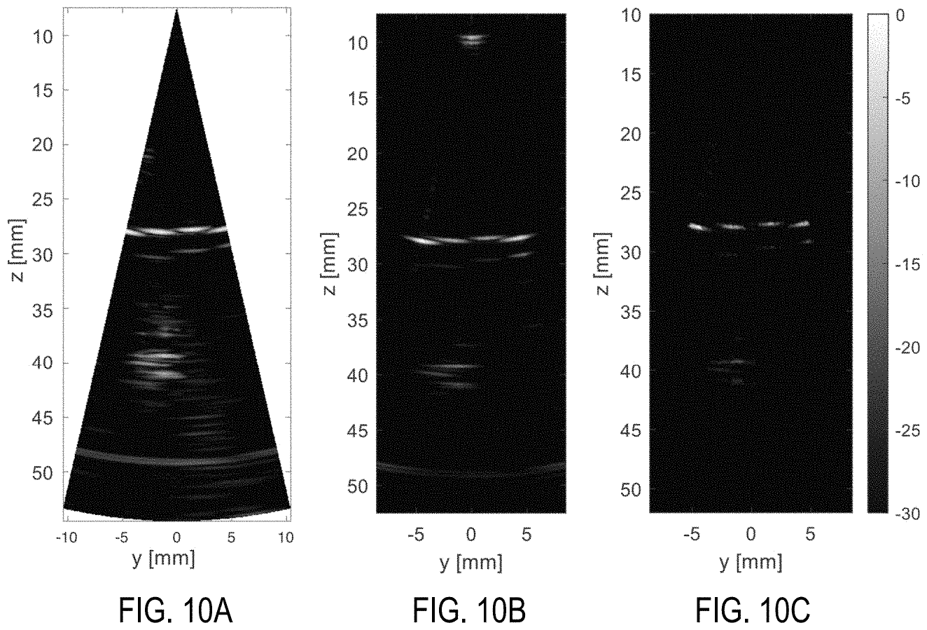

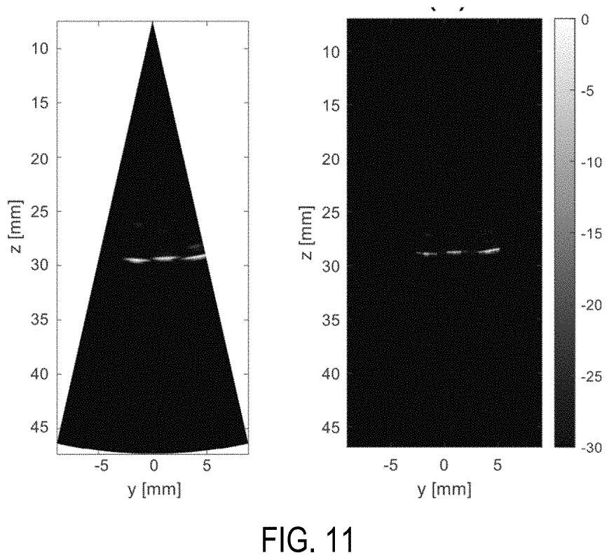

FIGS. 10A-10C show an example of using ultrasound data acquired from multiple spatial locations using multiple different ultrasound beams to beamform a higher quality image. As compared to FIG. 10A, it can be seen that the main lobe width in FIG. 10B gets reduced and the side lobe level gets suppressed when data acquired from multiple spatial locations are used for beamforming. Additionally, adaptive beamforming methods (e.g., minimum variance and generalized coherence factor) can be utilized to further improve the elevational resolution. FIG. 10C shows such an example, where it can be seen that, as compared to FIG. 10B, the imaging quality of the wire targets was further improved with significantly reduced main lobe width and side lobe level. FIG. 11 shows another example of implementing the same multiple-beam method on a different ultrasound transducer.

Additionally or alternatively, elevational resolution and imaging quality can be improved by using the point spread function (“PSF”) in the elevational dimension to filter the FASTER 3D images. Based on the ultrasound beam profile and movement of the tilting reflector, the spatial varying and/or time varying PSF can be characterized. As a non-limiting example, a deconvolution filter based on the PSF can be applied on the FASTER 3D images to compensate for the deteriorated spatial resolution because of the movement of the tilting reflector and the lack of transmit focusing from a single transducer element.

As another non-limiting example, machine learning-based methods can be applied to improve elevational resolution based on the desired PSF. Ultrasound simulation and/or experimental data acquired from known objects (e.g., wire targets) can be used to train neural networks to recover high resolution images based on the known PSF (e.g., in simulation) or measured PSF (e.g., in experiment with wire targets). The trained neural network can be applied to either pre-beamform raw channel data or post-beamform ultrasound data to further improve the imaging quality of images obtained using the FASTER systems and techniques described in the present disclosure.

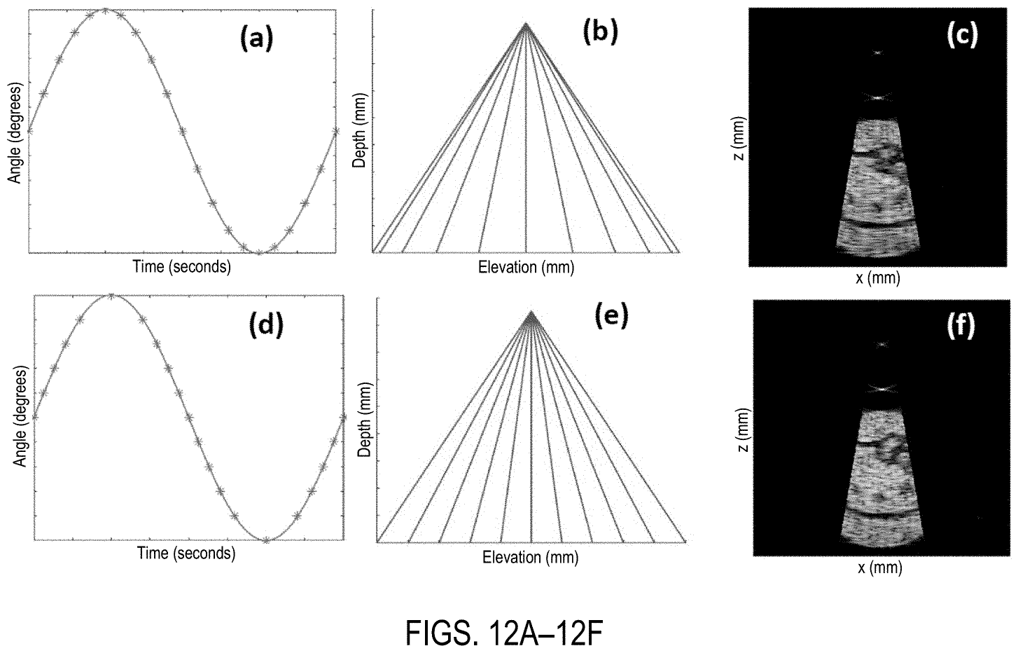

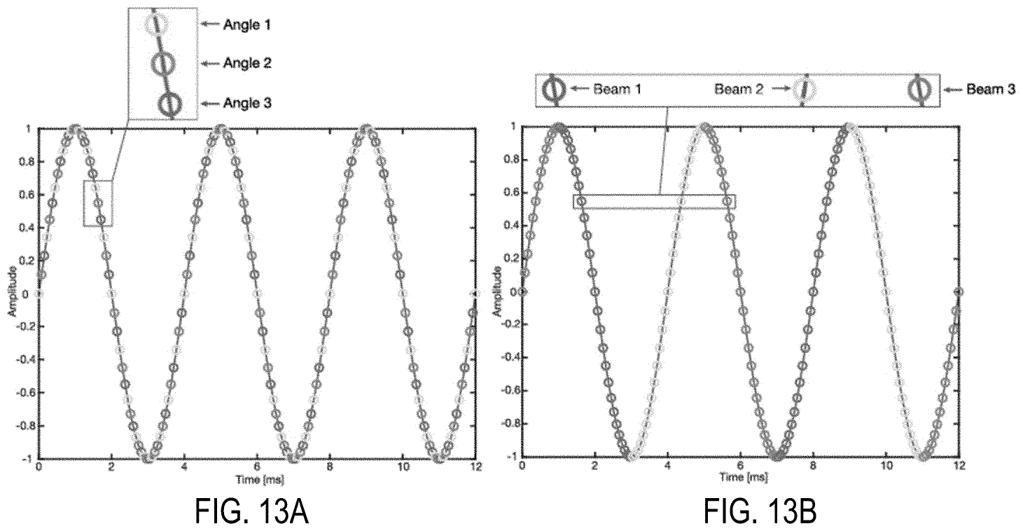

In an embodiment of the present disclosure, temporal sampling can be improved upon by adjusting the timing of the ultrasound data acquisition such that the spatial distribution of the scanning lines is homogeneous. As shown in FIGS. 12A-12C, as a non-limiting example, when the tilting reflector scans through a set of tilting angles using a sinusoidal tilting motion, then the spatial sampling will be uneven if the ultrasound sampling points are evenly sampled in time. This is illustrated in FIG. 12A as the lateral distance between the ultrasound sampling points being equal along the horizontal axis, which indicates time, but the vertical distance between the ultrasound sampling points is not equal along the vertical axis, which indicates the reflector tilting angle. As a result, the scan lines will be unevenly distributed in space, as shown in FIG. 12B. Consequently, the final reconstructed image will have coarser imaging pixel resolution towards the center of the image than that towards the lateral edges of the image, as shown in FIG. 12C. To achieve a homogeneous lateral imaging resolution, the timing of the ultrasound data acquisition can be adjusted such that the vertical distance between the ultrasound sampling points is equal, as shown in FIG. 12D. This may result in a non-evenly distributed temporal sampling pattern, but this technique results in the scan lines being evenly distributed in space, as shown in FIG. 12E, and contributes to a homogeneous imaging pixel resolution in the lateral direction, as shown in FIG. 12F.

The volume-rate of the 3D ultrasound imaging system is related to the pulse repetition frequency (“PRF”) of the ultrasound system 112, the number of pulse echoes to form an image slice (e.g., number of compounding angles in the compounding plane wave imaging, number of lines or focused beams in the focused beam line-by-line scanning), and tilting frequency of the tilting reflector. As a non-limiting example, if the spatial angular compounding imaging is used, then the effective pulse repetition frequency, PRFₑ:

$${PRF}_{e} = \frac{PRF}{{n}_{a}} ; \qquad ( 1 )$$

where nₐ is the number of compounding angles. When distributing the imaging planes along the elevational dimension via a tilting reflector, with a tilting frequency of Fₘ, the tilting angle (θₙ) of the tilting reflector with a sinusoidal driving signal corresponding to nth imaging plane is given by:

$$\begin{matrix}{θ}_{n} = A sin ( 2 π {F}_{m} {t}_{n} + ϕ ) + γ \\ = A sin ( 2 π {F}_{m} \frac{n}{{PRF}_{e}} + ϕ ) + γ ;\end{matrix} \qquad ( 2 )$$

where A is the half-side range of the tilting angle of the tilting reflector, tₙ is the time to sample the nth imaging plane (n=1, 2, . . . , Nₚ with n∈ [symbol] ⁺, where [symbol] ⁺ denotes a positive integer number), ϕ is the initial phase of the tilting reflector, and y is the tilting angle offset of the tilting reflector. Since the incident angle is equal to the reflection angle of an acoustic wave, the scanning angle (αₙ) of the tilting reflector is twice of the tilting angle (e.g., scanning angle is changed by 90 degrees when reflector is tilted by 45 degrees):

$${α}_{n} = 2 {θ}_{n} = 2 A sin ( 2 π {F}_{m} \frac{n}{{PRF}_{e}} + ϕ ) + 2 γ ; \qquad ( 3 )$$

The effective 3D imaging volume rate Fᵥ is given by:

$$\begin{matrix}{F}_{v} = \{ \begin{matrix}2 {F}_{m} & condition 1 \\ \frac{{F}_{m}}{m} & condition 2\end{matrix} ; \\ where , & ( 4 ) \\ condition 1 := ( \{ \frac{{PRF}_{e}}{{F}_{m}} ∈ {A}_{e} \} ⋂ \{ \frac{{PRF}_{e}}{{F}_{m} \cdot 2 π} ϕ ∈ {ℤ}^{+} \} ) ; & ( 5 )\end{matrix}$$

in which Aₑ denotes an even number, excluding imaging planes sampled at the largest scanning angles, condition 2 is the complement of condition 1, and in m∈ [symbol] ⁺ is the smallest number when (mPRFₑ/Fₘ)∈ [symbol] ⁺. The factor of two in condition 1 comes from the observation that each spatial location is imaged twice during one tilting cycle of the reflector. For example, if the PRFₑ=1062.5 Hz and Fₘ=250 Hz, then PRFₑ/Fₘ=4.25, which belongs to condition 2 with m=4. Therefore, the volume rate Fᵥ=62.5 Hz.

Subsequently, the number of imaging planes (Nₚ) sampled in one tilting cycle is:

$${N}_{p} = \frac{{PRF}_{e}}{{F}_{v}} + 1 \{ \{ {F}_{v} = 2 {F}_{m} \} ⋂ {C}^{*} \} ; \qquad ( 6 )$$

where C* indicates when imaging planes at the largest scanning angles were sampled, and 1{⋅} is the indicator function.

In embodiments of the present disclosure, the FASTER imaging device may be used with at least one of the imaging sequences that use plane wave imaging, compounding plane wave imaging, diverging beam imaging, compounding diverging beam imaging, focused beam imaging, wide beam imaging, synthetic aperture imaging, nonlinear imaging methods such as harmonic imaging, super-harmonic imaging, and ultra-harmonic imaging, and finally imaging methods with coded transmissions.

FIGS. 13A and 13B illustrate two methods to perform compounding plane wave imaging and focused beam imaging. As shown in FIG. 13A, compounding plane wave imaging is applied with different angles transmitted continuously. The volume rate stays the same with the volume rate when plane wave imaging is applied, while the number of sampled locations is reduced by a factor corresponding to the number of compounding angles. In FIG. 13B, line-by-line focused beam imaging is implemented with different angles transmitted in different sweeps. The volume rate is reduced by the factor of number of focused beams compared with the plane wave imaging case, and the number of sampled locations remains the same.

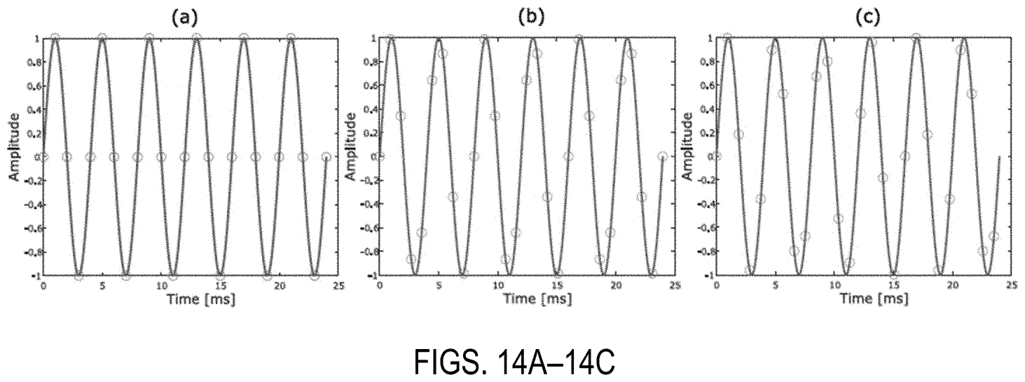

The volume rate may be reduced to achieve better image quality in elevational imaging plane. As one example, the tilting frequency of the tilting reflector can be reduced. In these instances, the voltage of the driving signal may need to be increased to alleviate the decrease of tilting angle range due to using a frequency that is off the resonant frequency of the tilting. As another example, the PRF can be changed to make the ratio of the PRF to the tilting frequency a non-integer number. As shown in FIGS. 14A-14C, this effectively lowers the imaging volume rate and increases the number of elevational sampling positions, which translates to a better imaging quality.

Since the 3D FOV is insonified by ultrasound beams that are reflected and swept by the tilting reflector assembly 104, which pivots on a central long axis, the raw ultrasound data are sampled on a polar coordinate (e.g., distance and angle from the origin). For display, the ultrasound data can be resampled on Cartesian coordinates, which can be achieved by interpolation or other suitable algorithms that can typically be used in scan conversions in ultrasound imaging (e.g., for imaging with the curved array transducers).

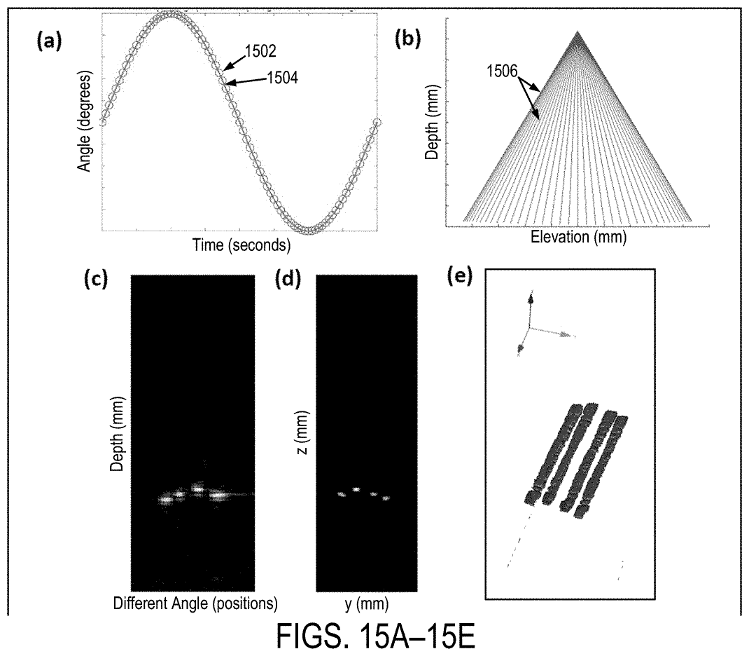

FIGS. 15A-15F illustrate a process for reconstructing an image (e.g., a 3D image) according to some embodiments of the present disclosure. Each circle 1502 in FIG. 15A represents a sampling data point that is temporally synchronized with the tilting reflector motion. The solid curve 1504 indicates the tilting reflector motion. In the spatial domain, each circle 1502, or data sampling point, relates to a scanning line 1506 in FIG. 15B. FIG. 15C shows the raw ultrasound data of the cross-sections of four thin wires before scan conversion, and FIG. 15D shows the ultrasound data of the same four wires after scan conversion. Note that the lateral dimension of the image changed from polar coordinates (FIG. 15C) to Cartesian coordinates (FIG. 15D). FIG. 15E shows the final reconstructed 3D images of the four wires, which uses volume rendering. Many other 3D visualization techniques can be utilized for FASTER 3D images, such as maximum intensity projection and isosurface visualizations.

As noted, when performing image reconstruction, the ultrasound data can be resampled from a polar coordinate to a Cartesian coordinate. Additionally or alternatively, the ultrasound data can be upsampled and aligned in space and in time, as described below in more detail. Advantageously, the ultrasound data can be any suitable type of ultrasound data, including ultrasound radiofrequency (“RF”) data, in-phase quadrature (“IQ”) data, processed ultrasound data, or combinations thereof. In this way, the systems and methods described in the present disclosure are capable of acquiring data and reconstructing images that include B-mode images, color-flow images, pulse wave Doppler signals, shear wave signals, blood flow signals, and tissue displacement signals.

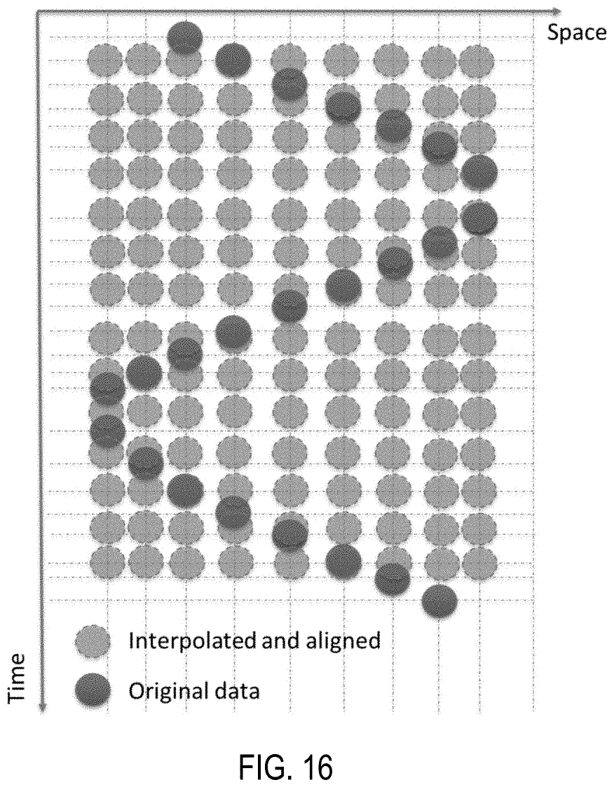

Because FASTER 3D imaging achieves 3D sampling by rapidly sweeping a ultrasound beam (e.g., unfocused plane waves) in the elevational direction, the sampling of the 3D FOV may not be continuous, both in time and in space. This is illustrated in FIG. 16, where it can be seen that only a subset of the continuous spatiotemporal data (orange circles in FIG. 16) are sampled. To recover the missing data, in an embodiment of the present disclosure, interpolation (e.g., 1D, 2D, or 3D interpolations) can be performed on either the spatial domain, the temporal domain, or both. Such interpolation can be performed on raw unbeamformed ultrasound data, on beamformed ultrasound data, or on processed ultrasound data such as blood flow signals and shear wave signals.

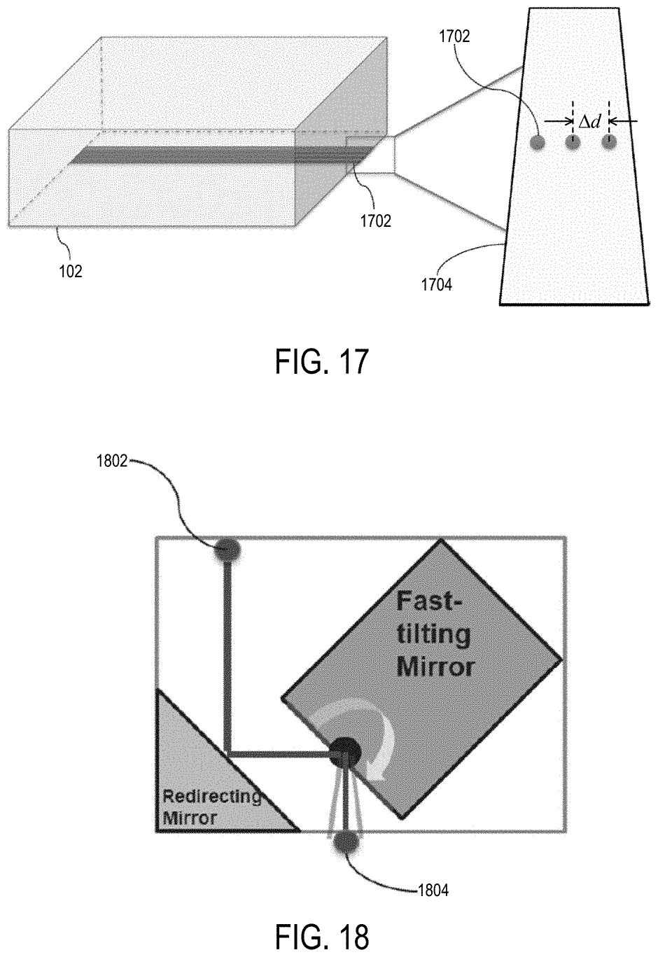

FIG. 17 illustrates a method for calibrating the FASTER imaging device 100 and/or corresponding reconstruction algorithm(s) used for 3D data reconstruction. For long-term use of the FASTER imaging device 100, the tilting reflector assembly 104 may need to be calibrated (e.g., driving voltage, driving frequency, reconstruction algorithms) for accurate 3D imaging. One calibration method can be based on wires that are integrated in the housing 102 of the FASTER imaging device 100, as shown in FIG. 17. In this example, a group of thin wires 1702 (e.g., two, three, or more wires) is attached to, or otherwise arranged within, the interior of the bottom part of the housing 102 where the ultrasound beams exit the housing 102 and enter the tissue (e.g., the acoustically transparent slot 204 in FIG. 2). The thin wires 1702 are positioned with a known distance in between them. For calibration, an image can be taken (illustrated as reference number 1704) and the distance, Δd, between the wires can be measured. The imaging performance can be tuned by adjusting the tilting reflector driving voltage, driving frequency, and the parameters used in 3D data reconstruction such as the tilting reflector scanning range and the time delay between the tilting reflector motion and ultrasound data acquisition. An optimal combination of parameters can give the correct distance measurements between the target wires 1702.

FIG. 18 shows another method that can be used to calibrate the FASTER imaging device 100 and corresponding reconstruction algorithm(s). This method is based on using a laser source 1802 and a position sensitive diode (“PSD”) detector 1804. As shown in FIG. 18, the laser source 1802 and the PSD detector 1804 are positioned so that the optical path between the laser source 1802 and the PSD detector 1804 overlaps with the acoustical path inside the housing 102 of the FASTER imaging device 100. For calibration, the PSD detector 1804 can measure the scanning range of the tilting reflector assembly 104 as well as the relative timing information between the input driving signal to the tilting reflector assembly 104 and the actual tilting reflector 116 position. Both information can be used in the reconstruction algorithm to facilitate accurate 3D reconstruction. The laser source 1802 and the PSD detector 1804 can be positioned towards one side of the housing 102 so that they are not interfering with acoustic wave propagation inside the housing 102. Both the laser 1802 and the PSD detector 1804 may share the same power supply and ways of communication with the tilting reflector assembly 104.

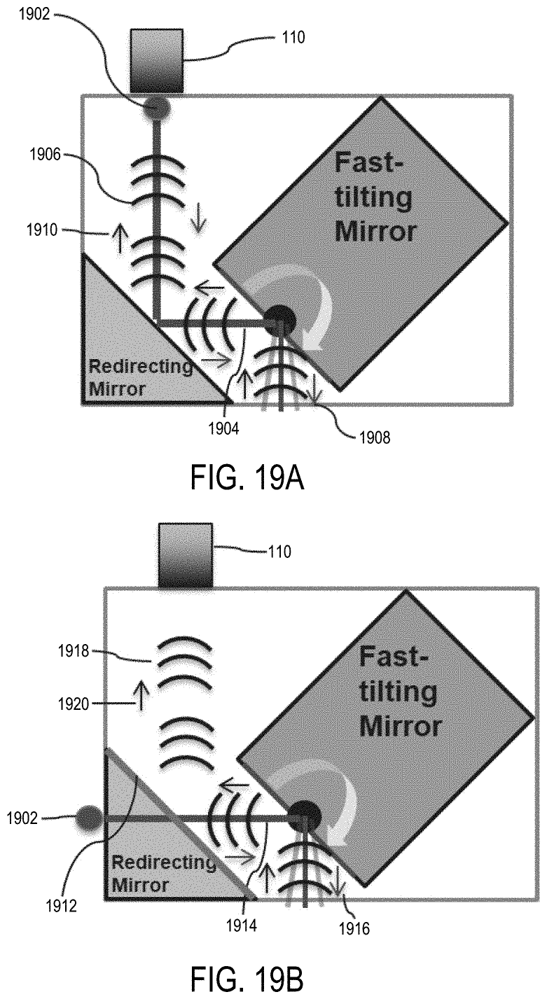

It should be noted that the described FASTER 3D imaging device can be used for 3D photoacoustic (PA) imaging. FIGS. 19A and 19B illustrate two non-limiting examples of 3D PA imaging in accordance with embodiments of the present disclosure. As shown in FIG. 19A, a laser source 1902 may be used to transmit pulsed laser into the tissue using the same mirror components inside the FASTER imaging device 100 (optical path shown by 1904 and propagation direction shown by 1908). The generated ultrasound signal (indicated by 1906) propagates back towards the ultrasound transducer 110 along direction 1910 using the same reflector components inside the FASTER imaging device 100. The ultrasound transducer 110 may be made optically transparent to facilitate the laser excitation and ultrasound reception.

Alternatively, as shown in FIG. 19B, the laser source 1902 may be positioned on the side of the FASTER imaging device 100. An optically-transparent acoustic reflector 1912 may be used in this case so that the laser can pass through the redirecting reflector 106 and then reflected and steered by the tilting reflector assembly 104 to illuminate different parts of the tissue. 1914 and 1916 indicate the optical wave propagation path and direction. The generated PA signal (indicated by 1918 and 1920) propagates back towards the ultrasound transducer 110.

For either case, the tilting reflector distributes the optical energy to different elevational positions of the tissue, therefore allowing 3D photoacoustic imaging.

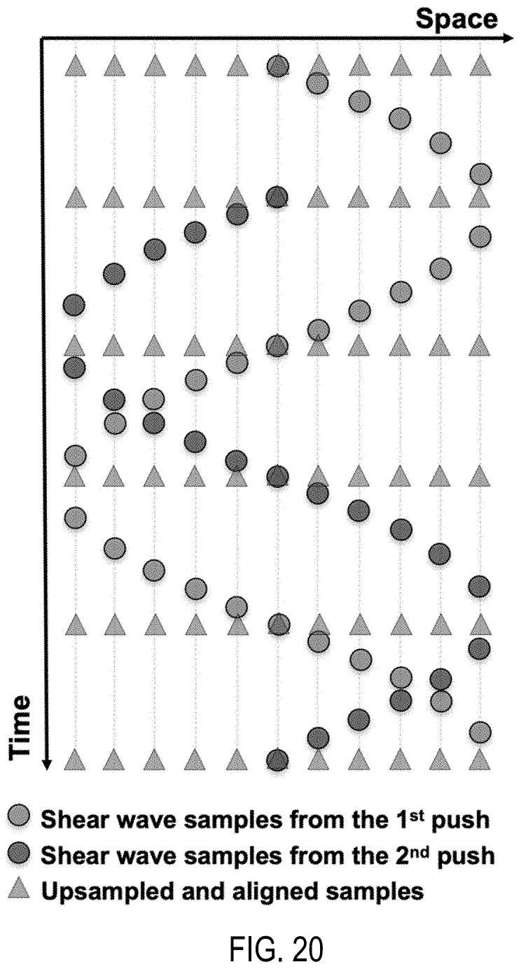

The FASTER imaging systems and techniques described in the present disclosure can also be used to achieve 3D shear wave elastography (“SWE”), both based on external vibration and acoustic radiation force (“ARF”)-induced shear waves. Because ARF-induced shear waves possess higher frequency components, it is advantageous to have a higher tracking volume rate to robustly track the 3D shear wave signal in these instances. To this end, a time-shifted and time-aligned sequential tracking method can be used to achieve such high 3D tracking rate. As illustrated in FIG. 20, to increase tracking volume rate, multiple shear wave push-detection cycles with various detection phase offsets can be used to generate shear wave data with adequate tracking volume rate. For example, as shown in FIG. 20, if two push-detection cycles are used, the detection of shear wave samples from the second push can be time-shifted by a quarter of the reflector tilting period, which effectively increases scanning volume rate by a factor of two when combined with shear wave samples acquired from the first push beam.

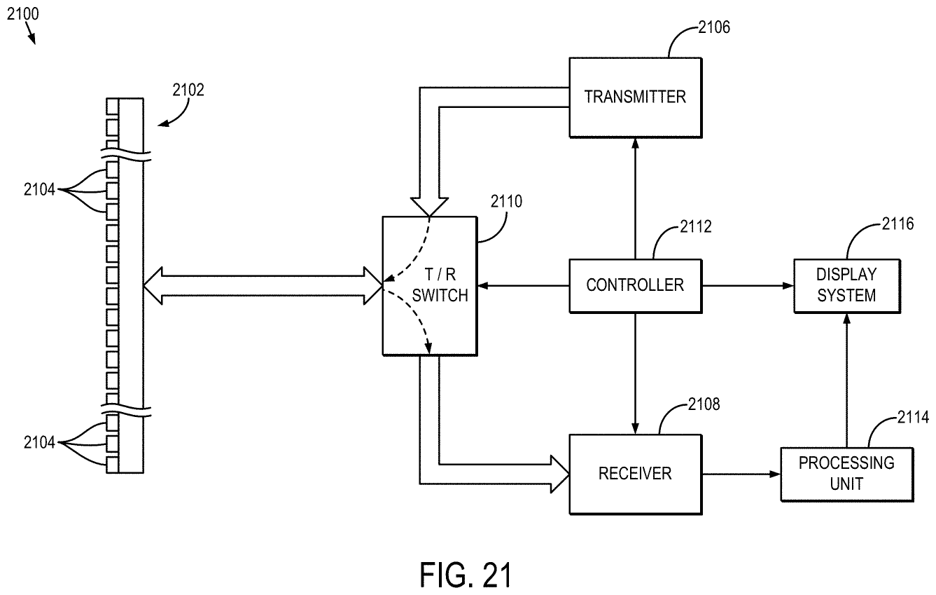

FIG. 21 illustrates an example of an ultrasound system 2100 that can implement the methods described in the present disclosure. The ultrasound system 2100 includes a transducer array 2102 that includes a plurality of separately driven transducer elements 2104. The transducer array 2102 can include any suitable ultrasound transducer array, including linear arrays, curved arrays, phased arrays, and so on. Similarly, the transducer array 2102 can include a 1D transducer, a 1.5D transducer, a 1.75D transducer, a 2D transducer, a 3D transducer, and so on.

When energized by a transmitter 2106, a given transducer element 2104 produces a burst of ultrasonic energy. The ultrasonic energy reflected back to the transducer array 2102 (e.g., an echo) from the object or subject under study is converted to an electrical signal (e.g., an echo signal) by each transducer element 2104 and can be applied separately to a receiver 2108 through a set of switches 2110. The transmitter 2106, receiver 2108, and switches 2110 are operated under the control of a controller 2112, which may include one or more processors. As one example, the controller 2112 can include a computer system.

The transmitter 2106 can be programmed to transmit unfocused or focused ultrasound waves. In some configurations, the transmitter 2106 can also be programmed to transmit diverged waves, spherical waves, cylindrical waves, plane waves, or combinations thereof. Furthermore, the transmitter 2106 can be programmed to transmit spatially or temporally encoded pulses.

The receiver 2108 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 2106 and the receiver 2108 can be programmed to implement a high frame rate. For instance, a frame rate associated with an acquisition pulse repetition frequency (“PRF”) of at least 100 Hz can be implemented. In some configurations, the ultrasound system 2100 can sample and store at least one hundred ensembles of echo signals in the temporal direction.

The controller 2112 can be programmed to design an imaging sequence using the techniques described in the present disclosure, or as otherwise known in the art. In some embodiments, the controller 2112 receives user inputs defining various factors used in the design of the imaging sequence.

A scan can be performed by setting the switches 2110 to their transmit position, thereby directing the transmitter 2106 to be turned on momentarily to energize transducer elements 2104 during a single transmission event according to the designed imaging sequence. The switches 2110 can then be set to their receive position and the subsequent echo signals produced by the transducer elements 2104 in response to one or more detected echoes are measured and applied to the receiver 2108. The separate echo signals from the transducer elements 2104 can be combined in the receiver 2108 to produce a single echo signal.

The echo signals are communicated to a processing unit 2114, which may be implemented by a hardware processor and memory, to process echo signals or images generated from echo signals. As an example, the processing unit 2114 can be configured to operate the acoustic steering device(s) described in the present disclosure (e.g., by controlling the tilting of the tilting reflector(s), controlling operation of the ultrasound transducer, controlling the synchronization between the tilting reflector(s) and the ultrasound transducers, and so on). Images produced from the echo signals by the processing unit 2114 can be displayed on a display system 2116.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), Flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. An acoustic steering device, comprising:
a housing;
a first reflector arranged within the housing;
a second reflector arranged within the housing and relative to the first reflector such that ultrasound beams incident upon the first reflector are reflected onto the second reflector whereupon the ultrasound beams are reflected to exit the housing; and
wherein at least one of the first reflector and the second reflector are tiltable and configured to tilt over a range of tilt angles responsive to a driving signal.

2. The acoustic steering device of claim 1, wherein the first reflector is tiltable such that when tilted over the range of tilt angles the ultrasound beams incident upon the first reflector are reflected onto the second reflector along different directions.

3. The acoustic steering device of claim 1, wherein the second reflector is tiltable such that when tilted over the range of tilt angles the ultrasound beams incident upon the second reflector are reflected out of the housing along different directions.

4. The acoustic steering device of claim 1, wherein:
the first reflector is tiltable such that when tilted over the range of tilt angles the ultrasound beams incident upon the first reflector are reflected onto the second reflector along different directions; and
the second reflector is tiltable such that when tilted over the range of tilt angles the ultrasound beams incident upon the second reflector are reflected out of the housing along different directions.

5. The acoustic steering device of claim 4, wherein the first reflector and the second reflector are coordinated to tilt over the range of tilt angles in order to enlarge of field-of-view of the ultrasound beams reflected by the second reflector.

6. The acoustic steering device of claim 1, wherein the at least one of the first reflector and the second reflector is configured to tilt over the range of tilt angles at an angular speed in a range of 250-500 Hz such that the ultrasound beams are steered to different positions at a high volume rate in a range of 500-1000 Hz.

7. The acoustic steering device of claim 1, at least one of the first reflector and the second reflector comprises a micro-fabricated mirror.

8. The acoustic steering device of claim 7, wherein the micro-fabricated mirror comprises a silicon mirror.

9. The acoustic steering device of claim 1, wherein at least one of the first reflector and the second reflector comprise a reflective mirror mounted on a rotational axle, and further comprising a micro-electrical motor configured to drive the rotational axle to tilt the reflective mirror through the range of tilt angles.

10. The acoustic steering device of claim 9, wherein the reflective mirror comprises one of a single-facet reflective mirror and a multi-facet reflective mirror.

11. The acoustic steering device of claim 9, wherein the rotation axle comprises a first hinge pair coupled to an external periphery of a frame and a second hinge pair coupling an inner periphery of the frame to the reflective mirror.

12. The acoustic steering device of claim 11, wherein the first hinge pair and the second hinge pair are independently controllable via driving currents with different frequencies.

13. The acoustic steering device of claim 11, wherein the first hinge pair has a higher bending stiffness and lower torsional stiffness than the second hinge pair.

14. The acoustic steering device of claim 1, wherein at least one of the first reflector and the second reflector is mounted on hinges that allow the at least one of the first reflector and the second reflector to tilt.

15. The acoustic steering device of claim 1, wherein at least one of the first reflector and the second reflector comprises:
a solenoid;
a micro-fabricated mirror comprising a mirror suspended on top of the solenoid;
magnets positioned on a backside of the micro-fabricated mirror such that the micro-fabricated mirror tilts in response to an input frequency and amplitude of the driving signal to the solenoid.

16. The acoustic steering device of claim 1, wherein the housing comprises an upper surface and a lower surface defining a volume therebetween, wherein the first reflector and the second reflector are arranged within the housing such that the ultrasound beams are incident upon the first reflector through the upper surface of the housing and the ultrasound beams incident upon the second reflector are reflected to exit the housing through the lower surface of the housing.

17. The acoustic steering device of claim 16, wherein the housing further comprises sidewalls such that the volume is an enclosed volume.

18. The acoustic steering device of claim 17, wherein the volume is filled with an acoustic conduction medium.

19. The acoustic steering device of claim 18, wherein the acoustic conduction medium comprises at least one of water, gel, or oil.

20. The acoustic steering device of claim 1, further comprising an acoustic lens positioned to focus the ultrasound beams.

21. The acoustic steering device of claim 1, further comprising an acoustic lens arranged relative to at least one of the first reflector and the second reflector such that ultrasound beams incident upon the acoustic lens from the at least one of the first reflector and the second reflector are focused onto a focal point.

22. The acoustic steering device of claim 21, wherein the acoustic lens is arranged such that ultrasound beams reflected from the second reflector are incident upon the acoustic lens.

23. The acoustic steering device of claim 1, wherein at least one of the first reflector and the second reflector have a curved surface such that ultrasound beams reflected from the curved surface are focused onto a focal point.

24. The acoustic steering device of claim 1, wherein the housing is composed of an acoustically transparent material.

25. The acoustic steering device of claim 1, further comprising a power source and a signal generator that are operable to generate the driving signal to drive the at least one of the first reflector and the second reflector to tilt over the range of tilt angles.

26. The acoustic steering device of claim 1, further comprising an ultrasound transducer configured to transmit the ultrasound beams to the first reflector and to receive ultrasound data corresponding to ultrasound beams reflected to the ultrasound transducer from the first reflector.

27. A three-dimensional ultrasound imaging system, comprising:
an ultrasound transducer configured to receive a driver signal from an ultrasound system and generate an ultrasound beam in response thereto;
a housing;
a tilting reflector arranged within the housing;
a redirecting reflector arranged within the housing;
a connector configured to couple the ultrasound transducer to the housing; and
wherein the tilting reflector is configured to tilt through a range of tilt angles in order to steer ultrasound beams incident upon the tilting reflector towards the redirecting reflector where the ultrasound beams are reflected by the redirector reflector to exit the housing.

28. The three-dimensional ultrasound imaging system of claim 27, wherein the connector is configured to receive a synchronization signal from the tilting reflector and to transmit the synchronization signal to the ultrasound transducer in order to synchronize the ultrasound transducer while the tilting reflector is tilted through the range of tilt angles.

29. A method for generating a three-dimensional image using an ultrasound system and an acoustic steering device coupled to the ultrasound system, the method comprising:
(a) transmitting ultrasound beams to a volume-of-interest using the ultrasound system while controlling the acoustic steering device to scan the ultrasound beams over a range of tilt angles;
(b) acquiring ultrasound data with the ultrasound system in response to the ultrasound beams transmitted to the volume-of-interest;
(c) reconstructing an image of the volume-of-interest using the computer system, wherein reconstructing the image includes associating beam positions of the ultrasound beams with tilting angles in the range of tilt angles.

30. The method of claim 29, wherein reconstructing the image includes performing a scan conversion on the ultrasound data.

31. The method of claim 29, wherein reconstructing the image includes beamforming using ultrasound data acquired from multiple different spatial locations in order to reconstruct the image to have increased elevational resolution.

32. The method of claim 29, wherein reconstructing image includes implementing at least one of adaptive beamforming of the ultrasound data or inputting the ultrasound data to a trained machine learning algorithm in order to reconstruct the image to have increased elevational resolution.
